SEQUENCE LISTING (2 sequences)

SEQ ID NO: 1 - DNA, 567 bp, rabbit
atggaggccctgccccttctggaaaacctaaccatttccgacattaatccctacgggttcacagtttcctggatggcatcggagaatgcctttgacagctttctagtaacggtggtggattctgggaagctgctggacccccaggaattcacactttcaggaacccagaggaagctggagcttagaggcctcataactggcattggctatgaggttatggtcctctggcttcacccaagggcatcaaacaagcccttgagggctgagattgttacagaagccgaaccggaagttgacaaccttctggtttcagatgccaccccagacggtttccgtctgtcctggacagctgatgaaggggtcttcgacaattttgttctcaaaatcagagataccaaaaagcagtctgagccactggaaataaccctacttgcccccgaacgtaccagggacttaacaggtctcagagaggctactgaatacgaaattgaactctatggaataagcaaaggaaggcgatcccagacagtcagtgctatagcaacaacacatcatcatcatcatcat

SEQ ID NO: 2 - protein, 189 aa, rabbit
MEALPLLENLTISDINPYGFTVSWMASENAFDSFLVTVVDSGKLLDPQEFTLSGTQRKLELRGLITGIGYEVMVLWLHPRASNKPLRAEIVTEAEPEVDNLLVSDATPDGFRLSWTADEGVFDNFVLKIRDTKKQSEPLEITLLAPERTRDLTGLREATEYEIELYGISKGRRSQTVSAIATTHHHHHH

(19) United States
(12) Patent Application Publication
(10) Pub. No.: US 2008/0145875 A1
(43) Pub. Date: Jun. 19, 2008

(54) ANTI-TENASCIN MONOCLONAL ANTIBODY IMMUNOASSAYS AND DIAGNOSTIC KITS

(75) Inventors: Darell D. Bigner, Mebane, NC (US); Charles N. Pegram, Hillsborough, NC (US); Chien-Tsun Kuan, Cary, NC (US)

Correspondence Address: DARBY & DARBY P.C., P.O. BOX 770, Church Street Station, New York, NY, 10008-0770, US

(73) Assignee: Duke University, Durham, NC (US)

(21) Appl. No.: 12/035,328

(22) Filed: Feb. 21, 2008

Related U.S. Application Data
(63) Continuation of application No. 11/282,117, filed on Nov. 16, 2005, now abandoned.
(60) Provisional application No. 60/628,940, filed on Nov. 17, 2004.

(51) Int. Cl. G01N 33/53

(52) U.S. Cl. 435/7.23

(57) ABSTRACT

The present invention provides immunoassays for detecting a tumor in a subject, comprising producing an antibody that specifically binds to tenascin, contacting the antibody with a biological sample suspected of containing tumor cells and determining the binding of the antibody to the biological sample. The present invention further provides methods of identifying a subject for treatment of a tumor. Kits for direct or indirect immunohistochemical or immunocytochemical assays are also provided. A novel polyclonal antibody that binds to tenascin domain TNfn C-D is further provided.

This application is a continuation application of U.S. patent application Ser. No. 11/282,117, filed Nov. 16, 2005 which claims the benefit of U.S. Provisional Patent Application No. 60/628,940, filed Nov. 17, 2004, which are hereby incorporated by reference in their entirety.

### GOVERNMENT SUPPORT

This invention was made with Government support under grant numbers MO1-RR 30, NS20023, CA11898, CA70164, CA42324, 1P50CA108786-01, 5P20CA96890 and PDT-414 from the National Center for Research Resources General Clinical Research Centers Program and National Institutes of Health. The Government has certain rights to this invention.

### FIELD OF THE INVENTION

The present invention concerns immunoassays and kits for the analysis of tissue samples and the detection and diagnosis of tumors and cancers.

### BACKGROUND OF THE INVENTION

Tenascin is a polymorphic extracellular matrix glycoprotein that is over-expressed in a variety of tumors including gliomas, melanomas and breast carcinomas. See Bourdon et al., Cancer Res. 43: 2796-2805 (1983); Howeedy et al., Lab. Invest. 63: 798-806 (1990); and Mackie et al., Proc. Natl. Acad. Sci. USA. 84: 4621-4625 (1987).

Bigner et al., U.S. Pat. No. 5,624,659, describes methods of treating solid and cystic tumors with monoclonal antibody 81C6. See also D. Bigner et al., J. Clin. Oncol. 16:2202-2212 (1998).

Rizzieri et al., U.S. patent application Ser. No. 10/008,062 (Publication No. US-2002-0187100-A1) describes anti-tenascin monoclonal antibody therapy for the treatment of lymphoma. See also D. Rizzieri et al., Blood 104, 642-648 (2004) (prepublished online Apr. 20, 2004); G. Akabani, G. et al., Int. J. Radiat. Oncol. Biol. Phys. 46:947-958 (2000).

Abrams et al., U.S. Pat. No. RE 38,008, concerns methods of improved cell targeting of antibody, antibody fragments, hormones and other targeting agents, and conjugates thereof.

There is, however, a need for specific, immunoassays and diagnostic kits for the analysis of tissue samples and the detection and diagnosis of tumors and cancers as well methods that provide an indication of potential patient response to therapy for the treatment of tumors and cancers.

### SUMMARY OF THE INVENTION

A first aspect of the invention relates to an immunoassay for detecting a tumor in a subject, comprising producing an antibody that specifically binds to tenascin, contacting the antibody with a biological sample suspected of containing tumor cells and determining the binding of the antibody to the biological sample. The antibody that binds to tenascin can be selected from the group consisting of monoclonal antibody 81C6 and an antibody that binds to the epitope bound by monoclonal antibody 81C6. The antibody that binds to tenascin can further be an antibody that specifically binds to tenascin domain TNfn C-Dhis.

A further aspect of the invention relates to a method of identifying a subject for treatment of a tumor comprising contacting an antibody that specifically binds to tenascin with a biological sample suspected of containing tumor cells, determining the binding of the antibody to the biological sample and assessing the overexpression of tenascin, wherein assessment of tenascin overexpression indicates that the subject is a candidate for treatment of a tumor comprising administering an antibody selected from the group consisting of monoclonal antibody 81C6 and an antibody that binds to the epitope bound by monoclonal antibody 81C6.

Additional aspects of the present invention relate to kits for a direct immunohistochemical or immunocytochemical assay comprising (a) an antibody that specifically binds to tenascin, the antibody labeled with a detectable group, and (b) instructions for use thereof in the immunohistochemical or immunocytochemical assay.

Further aspects of the present invention relate to kits for an indirect immunohistochemical or immunocytochemical assay comprising (a) a primary antibody that specifically binds to tenascin, (b) a secondary antibody that specifically binds to the primary antibody, wherein the secondary antibody is labeled with a detectable group, and (c) instructions for use thereof in the indirect immunohistochemical or immunocytochemical assay.

In still further aspects of the present invention, for the kits described herein, the extent of binding of the antibody to tenascin can be used to detect the presence of a tumor in a subject or identify subjects for treatment of a tumor comprising administering an antibody selected from the group consisting of monoclonal antibody 81C6 and antibodies that bind to the epitope bound by monoclonal antibody 81C6. Moreover, the kits can be packaged in a container and can also comprise control samples, wherein the control samples are positive, negative or both.

Additional aspects of the present invention relate to a novel antibody that specifically binds to tenascin domain TNfn C-Dhis.

The foregoing and other objects and aspects of the present invention are explained in detail in the drawings herein and the specification set forth below.

### BRIEF DESCRIPTION OF THE DRAWINGS

FIG. 1A presents a graphic illustration of the response from primary immunization using rabbit anti-TNfn C-D.

FIG. 1B presents a diagram illustrating the binding site of MAb 81C6 to tenascin.

FIG. 2 presents the cDNA (SEQ ID NO:1) and deduced amino acid sequence (SEQ ID NO:2) of TNfn C-Dhis.

### DETAILED DESCRIPTION OF THE EMBODIMENTS OF THE PRESENT INVENTION

It should be noted that as used herein and in the appended claims, the singular forms “a,” “and,” and “the” include plural referents unless the context clearly dictates otherwise.

Unless defined otherwise, all technical and scientific terms used herein have the same meaning as commonly understood to one of ordinary skill in the art to which this invention belongs. Although any methods, devices and materials similar or equivalent to those described herein can be used in the practice of the invention.

All publications mentioned herein are incorporated herein by reference to disclose and describe the methods and/or materials in connection with which the publications are cited.

The term “biological sample” as used herein refers to a fluid (blood, serum, urine, semen), intact cells or extracts thereof, or tissue samples. The biological sample may be a clinical cytology specimen (e.g., fine needle breast biopsy or pulmonary cytology specimen) or a human tissue specimen from, for example, stomach, lung, breast, ovarian, pancreatic, prostate or brain tumors. The tissue specimen may be fresh or frozen.

The terms “monoclonal antibody 81C6”, “antibody 81C6”, or similar terms encompass both the murine monoclonal antibody 81C6 and the humanized chimeric antibody 81C6, both of which are described in U.S. Pat. No. 6,624,659. Such monoclonal antibodies are produced in accordance with known techniques.

The term “antibodies” as used herein refers to all types of immunoglobulins, including IgG, IgM, IgA, IgD, and IgE. The term “immunoglobulin” includes the subtypes of these immunoglobulins, such as IgG₁, IgG₂, IgG₃, IgG₄, etc. Of these immunoglobulins, IgM and IgG are preferred, and IgG is particularly preferred. The antibodies may be of any species of origin, including (for example) mouse, rat, rabbit, horse, or human, or may be chimeric antibodies. See, e.g., M. Walker et al., Molec. Immunol. 26, 403-11 (1989). The term “antibody” as used herein includes antibody fragments which retain the capability of binding to a target antigen, for example, Fab, F(ab′)₂, and Fv fragments, and the corresponding fragments obtained from antibodies other than IgG. Such fragments are also produced by known techniques.

The term “polyclonal antibody” as used herein refers to multiple immunoglobulins in antiserum produced to an antigen following immunization, and which may recognize and bind to one or more epitopes to that antigen. Polyclonal antibodies used to carry out the present invention can be produced by immunizing a suitable subject of any species of origin, including (for example) mouse, rat, rabbit, goat, sheep, chicken, donkey, horse or human, with an antigen to which a monoclonal antibody to the target binds, collecting immune serum from the animal, and separating the polyclonal antibodies from the immune serum, in accordance with known procedures.

The term “primary antibody” as used herein refers to an antibody which binds specifically to the target protein antigen in a tissue sample. A primary antibody is generally the first antibody used in an immunohistochemical procedure. The primary antibody can be the only antibody used in an immunohistochemical procedure.

The term “secondary antibody” as used herein refers to an antibody which binds specifically to a primary antibody, thereby forming a bridge between the primary antibody and a subsequent reagent, if any. The secondary antibody is generally the second antibody used in an immunohistochemical procedure.

### 1. Subjects.

Subjects of the present invention include both human subjects for medical purposes and animal subjects for veterinary and drug screening and development purposes. Suitable animal subjects include both avians and mammals, with mammals being preferred. The term “avian” as used herein includes, but is not limited to, chickens, ducks, geese, quail, turkeys and pheasants. The term “mammal” as used herein includes, but is not limited to, primates, bovines, ovines, caprines, porcines, equines, felines, canines, lagomorphs, rodents (e.g., rats and mice), etc. Human subjects are the most preferred. Human subjects include fetal, neonatal, infant, juvenile and adult subjects.

Moreover, subjects described herein include subjects afflicted with or suspected of being afflicted with lymphoma, as well as subjects afflicted with or suspected of being afflicted with solid tumors or cancers such as lung, colon, breast, brain, liver, prostate, spleen, muscle, ovary, pancreas, skin (including melanoma), etc.

### 2. Antibodies.

The monoclonal antibodies of the present invention may be recombinant monoclonal antibodies produced according to the methods disclosed in Reading, U.S. Pat. No. 4,474,893, or Cabilly et al., U.S. Pat. No. 4,816,567. The antibodies may also be chemically constructed by specific antibodies made according to the method disclosed in Segel et al., U.S. Pat. No. 4,676,980. Applicants specifically intend that the disclosure of all U.S. patent references cited herein be incorporated herein by reference in their entirety.

Monoclonal antibodies may be chimeric antibodies produced in accordance with known techniques. For example, chimeric monoclonal antibodies may be complementarily determining region-grafted antibodies (or “CDR-grafted antibodies”) produced in accordance with known techniques.

Monoclonal Fab fragments may be produced in Escherichia coli by recombinant techniques known to those skilled in the art. See, e.g., W. Huse, Science 246, 1275-81 (1989).

As noted above, antibodies employed in carrying out the present invention are those which bind to tenascin. In some embodiments of the present invention, the antibody can be monoclonal antibody 81C6 or an antibody that binds to the epitope bound by monoclonal antibody 81C6 (i.e., antibodies that cross-react with, or block the binding of, monoclonal antibody 81C6). The monoclonal antibody 81C6 is a murine IgG2b monoclonal antibody raised from a hybridoma fusion following immunization of BALB/c mice with the glial fibrillary acidic protein (GFAP)-expressing permanent human glioma line U-251 MG, as known and described in M. Bourdon et al., Cancer Res. 43, 2796 (1983). In other embodiments of the present invention, the antibody can be a polyclonal antibody against a spliced variant of tenascin.

Particularly preferred for carrying out the present invention is a mouse-human chimeric monoclonal antibody 81C6, as described in U.S. Pat. No. 5,624,659 to Bigner and Zalutsky, or a rabbit polyclonal antibody, anti-TNfn C-D, as further described in the examples section below.

Antibodies for use in the present invention specifically bind to tenascin with a relatively high binding affinity, for example, with a dissociation constant of about 10⁻⁴ to 10⁻¹³. In embodiments of the invention, the dissociation constant of the antibody-tenascin complex is at least 10⁻⁴, preferably at least 10⁻⁶, and more preferably at least 10⁻⁹.

Antibodies of the present invention may be coupled to a radioisotope. The antibody can be coupled to a radioisotope using the techniques described in Current Protocols in Immunology, Volumes 1 and 2, Coligen et al., Ed. Wiley-Interscience, New York, N.Y., Pubs. (1991). Examples of radioisotopes which may be coupled to the antibody include, but are not limited to, ²²⁷Ac, ²¹¹At, ¹³¹Ba, ⁷⁷Br, ¹⁴C, ¹⁰⁹Cd, ⁵¹Cr, ⁶⁷Cu, ¹⁶⁵Dy, ¹⁵⁵Eu, 153 Gd, ¹⁹⁸Au, ³H, ¹⁶⁶Ho, ^{113m}In, ^{115m}In, ¹²³I, ¹²⁵I, ¹³¹I, ¹⁸⁹Ir, ¹⁹¹_{Ir,} ¹⁹²Ir, ¹⁹⁴Ir, ⁵²Fe, ⁵⁵Fe, ⁵⁹Fe, ¹⁷⁷Lu, ¹⁰⁹Pd, ³²P, ²²⁶Ra, ¹⁸⁶Re, ¹⁸⁸Re, ¹⁵³Sm, ⁴⁶Sc, ⁴⁷Sc, ⁷²Se, ⁷⁵Se, ¹⁰⁵Ag, ⁸⁹Sr, ³⁵S, ¹⁷⁷Ta, ¹¹⁷mSn, ¹²¹Sn, ¹⁶⁶Yb, ¹⁶⁹Yb, ⁹⁰Yt, ²¹²Bi, ¹¹⁹Sb, ¹⁹⁷Hg, ⁹⁷Ru, ¹⁰⁰Pd, ^{101m}Rh, and ²¹²Pb.

It will be appreciated that monoclonal antibodies as used herein incorporate those portions of the constant region of an antibody necessary to evoke the useful immunological response in the subject being affected.

### 3. Examples of Tumors, Cancers, and Neoplastic Tissue.

Examples of tumors, cancers, and neoplastic tissue that can be detected and/or diagnosed according to the present invention include, but are not limited to, malignant disorders such as breast cancers; osteosarcomas; angiosarcomas; fibrosarcomas and other sarcomas; leukemias; lymphomas (Hodgkin's lymphoma and Non-Hodgkin's lymphoma), and other blood cancers; myelodysplasia, myeloproliferative disorders; sinus tumors; ovarian, uretal, bladder, prostate and other genitourinary cancers; colon, esophageal and stomach cancers and other gastrointestinal cancers; lung cancers; myelomas; pancreatic cancers; liver cancers; kidney cancers; endocrine cancers; skin cancers; and brain or central and peripheral nervous system tumors, malignant or benign, including gliomas and neuroblastomas.

### 4. Immunohistochemistry.

Immunohistochemical (IHC) methods are well known by those skilled in the art. See, for example, U.S. Pat. No. 6,441,143 to Koski et al., U.S. Pat. No. 6,376,201 to Miron et al., U.S. Pat. No. 5,876,712 to Cheever et al., U.S. Pat. No. 5,854,009 to Klug, and U.S. Pat. No. 5,843,684 to Levine et al., U.S. Pat. No. 4,968,603 to Slamon et al. and “DAKO anti-Her2 IHC System for Immunoenzymatic Staining” (Package Insert) DAKO Corporation. As described in U.S. Pat. No. 6,573,043 to Cohen et al., two general methods of IHC are available: direct and indirect assays. According to the first assay, binding of an antibody to the target antigen is determined directly. This direct assay uses a labeled reagent, such as a fluorescent tag or an enzyme-labeled primary antibody, which can be visualized without further antibody interaction. The fluorescent tag or label can be fluorescein. The enzymatic label can be horseradish peroxidase or alkaline phosphatase.

In a typical indirect assay, unconjugated primary antibody binds to the antigen and then a labeled secondary antibody binds to the primary antibody. Where the secondary antibody is conjugated to an enzymatic label, a chromagenic or fluorogenic substrate can be added to provide visualization of the antigen. Such are described above. Signal amplification may occur because several secondary antibodies may react with different epitopes on the primary antibody. The primary and/or secondary antibody used for immunohistochemistry typically can be labeled with a detectable moiety. IHC techniques are further described in Immunohistochemical Staining Methods. Thomas Boenisch, ed. (3rd ed. 2001).

### EXAMPLES

The present invention will be better understood by reference to the following Examples, which are provided as exemplary of the invention, and not by way of limitation.

### Example 1

### Preparation of 81C6 Column

An 81C6 column was prepared according to the following protocol.

Weigh out CNBr activated Sepharose-4B, place into plastic centrifuge tube and swell in deionized water. One gram of dry Sepharose is 2-3 ml of swollen gel. Use 1 ml of gel for every 5 mg of 81C6 used. When gel is swollen remove water by centrifuging at 500×g. Discard supernatant and add 81C6 (1-2 mg/ml) in 115 mM Phosphate buffer, pH 7.4. Rock for two hours at room temperature and then overnight at 4° C. Remove non-bound 81C6 by centrifuging at 500×g. Save supernatant and read A₂₈₀ Calculate percent bound to Sepharose so you know total 81C6 bound. About 10 mg 81C6 bound is desired to bind 1 mg of Tenascin later. Wash Sepharose two more times and then add 1 M ethanolamine in 115 mM phosphate buffer and react for one hour at room temperature. Pour Sepharose into column and wash column with pH 11 Caps buffer and then with pH 3.5 citrate buffer (removes charged bound 81C6). Equilibrate column with 115 mM phosphate buffer and 0.5% Na Azide and store at 4° C.

### Example 2

### Preparation of Tenascin Column

A tenascin column was prepared according to the following protocol.

Weigh out CNBr activated Sepharose-4B, place into plastic centrifuge tube and swell in deionized water. One gram of dry Sepharose is 2-3 ml of swollen gel. Use 1 ml of gel for every milligram of tenascin used. When gel is swollen remove water by centrifuging at 500×g. Discard supernatant and add tenascin (0.1-1 mg/ml) in 0.1 M borate buffer, pH 8.5. Rock for two hours at room temperature and then overnight at 4° C. Remove non-bound tenascin by centrifuging at 500×g. Save supernatant and read A₂₈₀. Calculate percent bound to Sepharose so you know total tenascin bound. Wash Sepharose two more times and then add 1 M ethanolamine in 115 mM phosphate buffer and react for one hour at room temperature. Pour Sepharose into column and wash column with pH 11 Caps buffer and then equilibrate column with 115 mM phosphate buffer and 0.5% Na Azide and store at 4° C. It is preferred that acid pH buffer is not used on the tenascin column.

### Example 3

### Tenascin Immuno-Affinity Purification

Tenascin immunoaffinity purification was carried out according to the procedures set forth below.
1. Set up anti-tenascin (81C6) column and equilibrate with 115 mM PO₄ buffer (it should have been left in this buffer with 0.5% Na Azide).
2. Run through the 80CL3 (U-251MG CL3 Cell Line) supernatant. Save the supernatant flow through (pour into bottles and add 1 ml 10% Na azide per 500 ml bottle; store in refrigerator). One may need to pass flow through more than once to remove all tenascin. Approximately 1 to 3 μg/ml of culture supernatant is obtained.
3. Wash column with Tris-0.5 M NaCl buffer pH 8.0 (use about 300 ml for washing).
4. Set up 20 screw top, 1 ml plastic tubes with some solid glycine in the bottom to neutralize CAPS. These will be used to collect fractions off the column.
5. When wash is down to the level of the beads in the column, add 30 ml of pH 11.0 CAPS buffer to elute the column and collect 1 ml fractions in the prepared tubes (fill up to the ribbing on the neck of the tubes).
6. Re-equilibrate column in 0.115 PO₄ buffer. Wash through about 200 mls and then add 100 μl of 10% Na azide to the column, take off the tubing's, cap the ends of the tube (leaving the column full of the buffer and azide), and store it in the refrigerator.
7. Tenascin is dialyzed against pH 8.5 Borate Buffer for storage. Dialysis tubing should be soaked overnight in 1% Triton X-100 solution in deionized water and rinsed with deionized water prior to use. If Triton treated dialysis tubing is not used, all of the tenascin will bind to the tubing.
   If the column is not large enough to bind all the tenascin, the flow through can be passed over the column several times.

### Example 4

### Rabbit Anti-Tenascin Immuno-Affinity Purification

Rabbit anti-tenascin immuno-affinity purification was carried out according to the following procedure.
1. Set up Tenascin column and equilibrate with 115 mM PO₄ buffer (it should have been left in this buffer with 0.5% Na Azide).
2. Run through the rabbit anti-tenascin serum. Save the flow through; store in refrigerator).
3. Wash column with 0.115 PO₄ buffer (use about 300 ml for washing).
4. Set up 20 screw top, 1 ml plastic tubes with some solid glycine in the bottom to neutralize CAPS. These will be used to collect fractions off the column.
5. When wash is down to the level of the beads in the column, add 30 ml of pH 1.0 CAPS buffer to elute the column and collect 1 ml fractions in the prepared tubes (fill up to the ribbing on the neck of the tubes).
6. Re-equilibrate column in 0.115 PO₄ buffer. Wash through about 200 ml and then add 100 μl of 10% Na azide to the column, take off the tubing, cap the ends of the tube (leaving the column full of the buffer and azide), and store it in the refrigerator.
7. Read A₂₈₀ of fractions and repeat above steps with flow through until eluted fractions are negative for protein
8. Pool fractions containing antibody and dialyze against 115 mM phosphate buffer. Filter sterilize antibody into 2 ml sterile ampoules and store at 4° C.

### Example 5

### A. Tenascin Purification

Tenascin was initially purified from U-251 MG-C13 supernatant by immunoaffinity chromatography using the murine anti-tenascin MAb 81C6 (Bourdon et al., 1983). Culture supernatant was passed over an 81C6-Sepharose 4B affinity column at room temperature, the column was washed with 10 mM Tris plus 500 mM NaCl (pH 8.0), and the tenascin was eluted with 0.1 mM CAPS in 500 mM NaCl (pH 11.0) into tubes containing 30 ng of glycine per ml of eluate to neutralize the pH to approximately 8.3. Tenascin used for polyclonal antibody preparation was subjected to an additional glycerol gradient-sedimentation purification step (Erickson and Taylor, 1987).

### B. Production of Polyclonal Antisera

Polyclonal antiserum to tenascin was prepared against affinity purified tenascin. 5 μg of tenascin in Freund's complete adjuvant was injected s.c. into rabbits; nine subsequent monthly i.v. boosts of 5 p.g were administered, with high titers (1:50,000 against purified human tenascin) noted after the second boost. Antiserum from a bleed drawn 11 days after the second boost was used for all studies. No reactivity of this antiserum to ZO +10% FBS or to purified human fibronectin was noted on immunoblots (data not shown). See Ventimiglia J. B. et al., Journal of Neuroimmunology, 36 (1992) 41-55.

### Immunoaffinity Column Buffers

### Buffer (0.1M CAPS and 0.5M NaCl).

2.213 gms CAPS.

2.922 gms NaCl.

Dissolve in 100 ml of deionized water and adjust pH to 11.0 with HCl.

Glycine-HCl buffer pH 3.0.

41.83 gms glycine.

8.5 gms NaCl.

8.3 ml concentrated (12 N)HCl.

Dissolve in 1000 ml deionized H₂0, check pH and adjust to 3.0.

### 1M Tris-buffer pH 8.0 or 9.0.

60.55 gms Tris base.

Q.S. to 500 ml with deionized H₂O.

Adjust pH with HCl to pH 8.0 or 9.0.

Tris-0.15M NaCl buffer.

8.5 gms NaCl.

10 ml 1.0 M Tris-buffer pH 8.0.

Q.S. to 1000 ml with deionized H₂O.

Tris-0.5M NaCl buffer.

22.13 gms NaCl.

10 ml 1.0 M Tris-buffer pH 8.0. Q.S. to 1000 ml with deionized H₂O

Phosphate Buffer (0.115M phosphate). 5× stock concentrate.

90.65 gms NaH₂PO₄

373.05 gms Na₂HPO₄

Q.S. to 6 liters with deionized H₂O and pH should be between 7.3 and 7.4

Dilute 1 part with 4 parts deionized H₂O for working solution.

### 0.1M Citrate Buffer pH 3.5.

0.1 M sodium citrate 29.4 gms/liter deionized H₂O.

0.1 M citric acid 21 gms/liter deionized H₂O.

Adjust pH of 0.1 M citric acid solution to pH 3.5 with 0.1M sodium citrate solution.

### pH 8.5 Borate Buffer

0.1M Na Borate

0.5M NaCl

Adjust pH to 8.5

### Example 6

### A. Purification of TNfn C-Dhis

Tenascin domain TNfn C-Dhis expressing E. coli were grown in superbroth in an orbital shaker at 37° C. until it reached an optical density at A₆₀₀ of 1.5 to 2.0. Proteins were then expressed by the addition of IPTG to a concentration of 1 mM. Cultures were incubated for another 90 minutes and centrifuged at 13,000×g for 10 minutes. Bacterial pellets were resuspended in 50 mM Tris and 0.5 M NaCl; pH 8.0 buffer (TBS). Thirty milliliters TBS was added per 10 gms of bacteria and pellet resuspended by homogenizing with a Virtis VirTishear homogenizer. Bacteria was frozen and thawed twice and then completely lysed by the addition of 0.2 g lysozyme per ml of bacteria suspension. After agitating for one hour at room temperature, DNase 1 (Sigma Chemical Co.) was added to the lysed bacteria at a concentration 2000 units per 10 gms to break up DNA. After incubating for 30 minutes at room temperature, the preparation was centrifuged for 30 minutes at 13,000×g. Pellets were resuspended in TBS with 1% Triton X-100 and agitated for one hour at room temperature and then centrifuged for 40 minutes at 22.000×g. Pellet was resuspended in TBS with 1% Triton X-100 and centrifugation repeated. Resuspensions and centrifugations were repeated until supernatant contained only trace amounts of protein. Pellets were resuspended in TBS with 6 M urea and agitated. TNfn C-Dhis was purified on a nickel-NTA silica HPLC column (Qiagen, cat #30710) both from the 1% Triton X-100 extract and the 6 M urea extract. The TNfn C-Dhis in 6 M urea was refolded on the nickel column by decreasing the urea concentration form 6 M to TBS without urea with a 2 hour linear gradient. TNfn C-Dhis was eluted from column with a 1 hour linear gradient from TBS to TBS plus 300 mM imidazole. Eluted protein was dialyzed against 115 mM phosphate buffer pH 7.4. Protein concentration determined by Lowry method and then aliquoted and stored frozen at −135° C. until needed.

### B. Rabbit Immunization Procedure

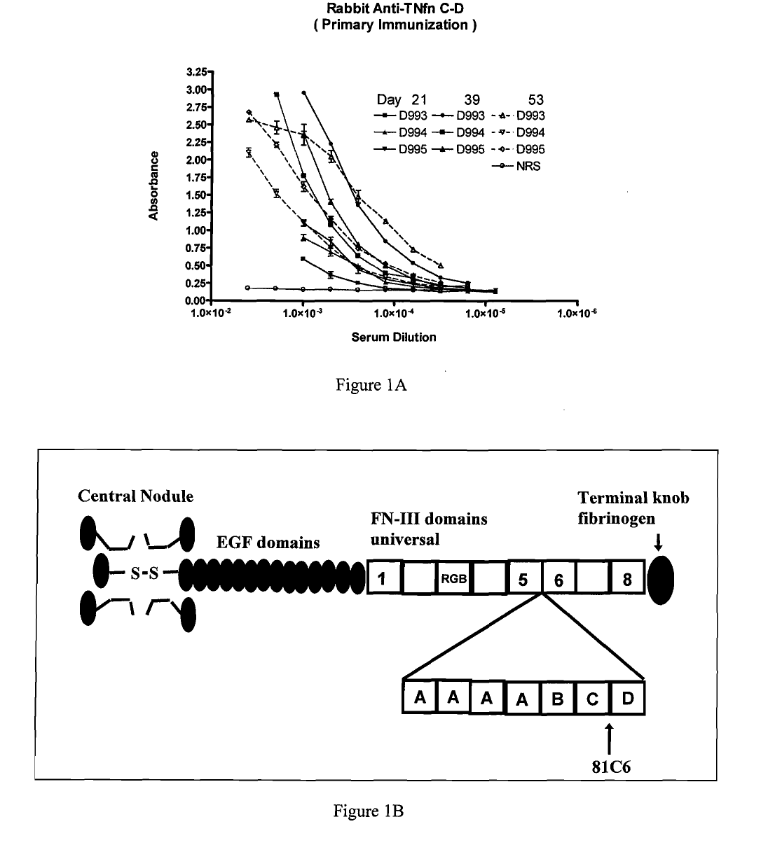

Rabbits were immunized with 100 μg of purified TNfn C-Dhis in complete Freund's adjuvant and a test bleed performed at days 21, 39 and 53. Since titer at day 53 had decreased they were boosted at day 60 with 50 μg TNfn C-Dhis in incomplete Freund's Adjuvant and were bled every 14 days until the titer starts to drop. The rabbits were then boosted with 50 μg TNfn C-Dhis in incomplete Freund's Adjuvant and bled every 14 days until titer started to drop. This procedure was repeated as needed. Titers were measured by ELISA against TNfn C-Dhis coated plates and response from primary immunization is shown in FIG. 1A.

### C. Purification of Rabbit Anti-TNfn C-D by Immunoaffinity Chromatography

An affinity column was made by coupling purified TNfn C-D through amine groups to a NHS activated-Sepharose 4 Fast Flow resin (Amersham, Cat # 17-0906-01). Rabbit antiserum was passed through a column, and the column was then rinsed with 10 column volumes of equilibrated buffer. Anti-TNfn C-D was eluted with 0.1 M CAPS buffer, pH 11.0. Eluted fraction were neutralized by the addition of powered glycine (5 mg/ml) to collection tube. Antibody was dialyzed against 115 mM phosphate buffer pH 7.4 and then filtered through a 0.22 μg filter into sterile vials. Protein concentration was determined by Lowry and vials were stored at 4° C. until used.

### P D. Plasmid Construction for TnfnCD Expression

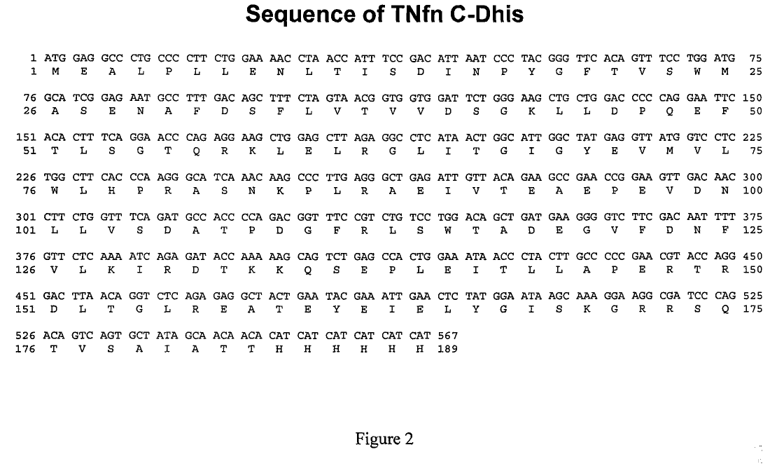

TNfnA-D bacterial expression plasmid vector was graciously provided by H. P. Erickson, Duke University, Durham, N.C. An NdeI site and ATG translation initiation codon were introduced at the 5′-end and a tag of six-Histidine cDNA sequence as well as a stop codon with EcoRI site were introduced at the 3′-end of the TNfn C-D cDNA fragment respectively by PCR using the TNfnA-D as the template. The resulting PCR fragment was used to clone into an expression vector pMR1scFv (Kuan et al., 1999), pre-cut by NdeI and EcoRI enzymes, to produce an expression plasmid. The cDNA and deduced amino acid sequence are shown in FIG. 2. The expression of TNfn C-D recombinant protein fragments was under the control of the T7 promoter. The recombinant protein was expressed in isopropyl thiogalactoside (IPTG)-induced E. coli BL21(λDE3) cells and accumulated in inclusion bodies. Bacterial cultures were inoculated into Superbroth containing 100 μg ampicillin/ml and grown at 37° C. to an OD₆₀₀ of 2.0-2.5. IPTG was added to 1 mM and growth was continued for 90 min. The cells were then sedimented by centrifugation and resuspended in 50 mM Tris-HCl, 20 mM EDTA, pH 7.4 for storage at −70° C.

### Example 7

### Anti-Tenascin Polyclonal Antibody Immunohistochemistry Protocol for Formalin Fixed, Paraffin Embedded Tissue

The following protocol can be employed to determine if rabbit anti-tenascin polyvalent/polyclonal antibody reacts with tenascin in patient samples, wherein tenascin is a large extracellular matrix protein in gliomas often associated with blood vessels.

### Specimen:

Formalin fixed patient brain tumor cut at 5-10 microns on slides, provided by Histology.
Needed: 6 slides, one section per slide, at 5-10 microns.

### Controls:

Formalin fixed D245 (human glioma tissue positive for tenascin, grown as rat xenograft).
Needed: 6 slides, one section per slide, at 5-10 microns.
Quality Control The following antibody must be run with anti-tenascin polyvalent serum in every assay:
1-Normal Rabbit IgG: Source: DAKO, #X0936. Beef liver powder and agarose absorbed as necessary; quantitation of rabbit IgG by Quantitative Capture ELISA required after absorption for determination of IgG concentration.

### Equipment:

### Slides (Fisher Scientific 12-550-15)

### Coverslips (VWR 48366067)

Glass staining dishes and trays (VWR 25445004)
Fume hood (when using xylenes)

### PAP Pen (Research Products International Corp.)

| Reagents: | Source | Concentration Used: |
|---|---|---|
| PBS | Dulbecco's 21600069 | Neat |
| Hydrogen Peroxide 30% | Sigma H-1009 | Used with MeOH |
| Methyl alcohol | Mallinckrodt AR 3016 | Neat |
| Ethyl alcohol (95 and 100%) | AAPER | Neat |
| Xylene | Mallinckrodt AR 8668 | Neat |
| Hematoxylin | Harris' Modified (Fisher) | Neat |
| DAB chromogen | Pierce 34065 (kit) | 10% in manufacturer's buffer |
| Normal goat serum | Zymed 01-6201 10 mls | 10% in DPBS |
| Biotinylated goat anti-rabbit | Zymed 65-6140 | 1/300 dilution in DPBS |
| serum(~IgG H + L) | | |
| HRP-Streptavidin | Zymed 43-4323 | 1/300 dilution in DPBS |
| Hemo-De | Fisher (15-182-507A) | Neat |
| Giemsa | Sigma GS-1L | 10% in dH₂O |
| May Grunwald | Sigma MG-1L | Neat |
| Mounting medium (Cytoseal) | VWR (48212-187) | Neat |
| ammonium hydroxide | Mallinckrodt (1177-4) | 1/6 dilution with dH₂O |

### Slide Set-Up:

| slide | speci- | | secondary | |
|---|---|---|---|---|
| # | men | primary ab dilution | reagent | tertiary reagent |
| 1 | D245 | PBS | none | HRP-SA @ 1/300 |
| 2 | | PBS | G~Rb @ 1/300 | ″ |
| 3 | | NRbIgG @ 5 μg/ml | ″ | ″ |
| 4 | | NRbIgG @ 2.5 μg/ml | ″ | ″ |
| 5 | | Rb~Ten @ 5 μg/ml | ″ | ″ |
| 6 | | Rb~Ten @ 2.5 μg/ml | ″ | ″ |
| 7 | Pa- | PBS | none | ″ |
| | tient | | | |
| | #1 | | | |
| 8 | | PBS | G~Rb @ 1/300 | HRP-SA @ 1/300 |
| 9 | | NRbIgG @ 5 μg/ml | ″ | ″ |
| 10 | | NRbIgG @ 2.5 μg/ml | ″ | ″ |
| 11 | | Rb~Ten @ 5 μg/ml | ″ | ″ |
| 12 | | Rb~Ten @ 2.5 μg/ml | ″ | ″ |
| 13 | Pa- | PBS | none | HRP-SA @ 1/300 |
| | tient | | | |
| | #3 | | | |
| 14 | | PBS | G~Rb @ 1/300 | ″ |
| 15 | | NRbIgG @ 5 μg/ml | ″ | ″ |
| 16 | | NRbIgG @ 2.5 μg/ml | ″ | ″ |
| 17 | | Rb~Ten @ 5 μg/ml | ″ | ″ |
| 18 | | Rb~Ten @ 2.5 μg/ml | ″ | ″ |

### Procedure:

To remove paraffin:
1) Soak slides 3×15 minutes in xylene baths
2) Soak 2×5 minutes in 100% ETOH
3) Soak 2×5 minutes in 95% ETOH
4) Air dry and encircle with PAP pen to make a well for the reagents

### Blocking:

1) Endogenous Peroxidase Block: soak for 10 min in MeOH/H₂O₂ solution (3 ml 30% H₂O₂ in 300 ml MeOH)
2) Rehydrate in DPBS for 10 min
3) Incubate for 30 min in 10% Normal Rabbit Serum (NRS)

### Immunohistochemistry:

1) Incubate with primary antibody over night @ 4C; approximately 0.2 ml/section, or appropriate to cover.
2) The next morning, allow slides to equilibrate to room temperature for at least one hour
3) Rinse with DPBS at an minimum rate of 2 mls/7 sec per section.
4) Incubate for 30 minutes at RT in 1/300 dilution of Zymed Biotinylated Goat anti Rabbit IgG serum in DPBS
5) Rinse with DPBS at same rate
6) Incubate for 10 minutes in 1/300 dilution of Zymed HRP-SA in DPBS at RT
7) Rinse with DPBS at same rate

### Stain/Counterstain:

1) Apply DAB (Chromogen) for 5 minutes or until brown staining appears in positive control slide. (Dilute DAB 1/10 in substrate buffer)
2) Rinse with DPBS
3) Soak 30 seconds in Harris' Modified Hematoxylin
4) Rinse in dH₂O
5) Wash in bluing agent (300 mls dH₂O with 6-8 drops of ⅙ diluted NH₄OH)
6) Rinse well in dH₂O
7) If case is melanotic, run “Giemsa Counter Staining”. See below *
8) Wash 2× in 95% ETOH baths
9) Wash 2× in 100% ETOH baths
10) Wash 3× in Hemo-De baths

### Mounting:

1) Coverslip with Cytoseal mounting media
2) Bake at 60 C for at least one day before storing

### *For Suspected Melanoma Cells, Counterstain Used is Giemsa

Giemsa Counter Staining (changes melanin from brown to green):
1) Incubate in May-Grunwald solution for 3 minutes at RT
2) Blot off excess stain
3) Incubate in Giemsa stain for 10 min at RT (Giemsa is to be diluted 1/10 in dH₂O)
4) Rinse in dH2O
5) Continue with alcohol and Hemo-De baths as of 8-10 above

### Example 8

### 81C6 Monoclonal Antibody

### Immunohistochemistry Protocol for Cytospins and Frozen sections

Positive Control Tissue: known glioma (D245MG rat xenograft)

### Positive Antibody Control: 3B4

Negative Reagent Control: DPBS, irrelevant murine IgG2b (M45.6), IgG1 (P588)
Negative Assay Controls: DPBS as 1° reagent

### Tenascin Detecting MAb: 81C6

### To Fix:

1) Fix in −20 C Acetone for 30 sec

### Immunohistochemistry:

1) Air dry and encircle with PAP pen to make well for reagents.
2) Endogenous Peroxidase Block: soak for 10 min in MeOH/H₂O₂ solution (3 ml 30% H₂O₂ in 300 ml MeOH)
3) Rehydrate in DPBS for 10 min
4) Incubate for 30 min in 10% normal serum from the species in which the secondary antibody was prepared (normal horse serum, Vector S-2000).
5) Incubate with 1′ antibody for 2 hrs at RT (MAb 81C6 (IgG2b), 3B4 (IgG1) and irrelevant IgG1 and IgG2b controls.
6) Rinse well with DPBS
7) Incubate for 60 min at RT in biotinylated secondary reagent (horse anti-mouse IgG, (Vector BA-2001) at 1/75- 1/150.
8) Rinse well with DPBS
9) Incubate for 10 min in 1/300 dilution of HRP-SA (Zymed 43-4323) in DPBS at R T
10) Rinse well with DPBS

### Stain/Counterstain:

1) Apply DAB (Chromogen) for 5 min or until brown staining appears in positive control slide. (Dilute DAB 1/10 in substrate buffer, Pierce System.)
2) Rinse well with DPBS
3) Soak 30 sec in Harris' Modified Hematoxylin
4) Rinse well in dH2O
5) Wash in bluing agent (300 ml dH2O with 6-8 drops 2N NH3)
6) Rinse well in dH2O
7) If case is melanotic, run “Giemsa Counter Staining”
8) Wash 2× in 95% ETOH baths
9) Wash 2× in 100% ETOH baths
10) Wash 3× in Hemo-De baths

### Mounting:

1) Coverslip with Surgipath micromount
2) Bake at 60 C for at least one day before storing

### Alternate Counterstain

1)*For suspected melanoma cells, counterstain used is Giemsa Giemsa Counter Staining (changes melanin from brown to green):
2) Incubate in May-Grunwald solution for 3 minutes at RT
3) Blot off excess stain
4) Incubate in Giemsa stain for 10 min at RT (Giemsa is to be diluted 1/10 in dH2O)
5) Rinse in dH2O
6) Continue with alcohol and Hemo-De baths as in 8-10 above

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

What is claimed is:

1. An immunoassay method for detecting a tumor in a subject, comprising:
(a) contacting a biological sample obtained from the subject with an anti-tenascin antibody;
(b) determining a level of binding of the anti-tenascin antibody to the biological sample;
(c) contacting a control non-tumor-bearing biological sample with the same anti-tenascin antibody as that used in step (a);
(d) determining a level of binding of the anti-tenascin antibody to the control non-tumor-bearing biological sample; and
(e) comparing the level of binding determined in step (b) with the level of binding determined in step (d), wherein an elevated level of binding of the antibody to the biological sample relative to the level of binding of the antibody to the control non-tumor-bearing control biological sample is indicative of the presence of the tumor; and further wherein
said anti-tenascin antibody is selected from the group consisting of polyclonal anti-tenascin antibody, murine monoclonal antibody 81C6 and fragments thereof, humanized chimeric monoclonal antibody 81C6 and fragments thereof, and an antibody that binds to the epitope bound by monoclonal antibody 81C6.

2. The immunoassay method of claim 1, wherein the subject is a human subject.

3. The immunoassay method of claim 1, wherein the biological sample is fluid, intact cell, cell extract or tissue.

4. The immunoassay method of claim 1, wherein the tumor is lymphoma.

5. The immunoassay method of claim 4, wherein the lymphoma is Hodgkin's lymphoma.

6. The immunoassay method of claim 4, wherein the lymphoma is Non-Hodgkin's lymphoma.

7. The immunoassay method of claim 1, wherein the antibody is coupled to a radioisotope.

8. The immunoassay method of claim 7, wherein the radioisotope is selected from the group consisting of ²²⁷Ac, ²¹¹At, ¹³¹Ba, ⁷⁷Br, ¹⁴C, ¹⁰⁹Cd, ⁵¹Cr, ⁶⁷Cu, ¹⁶⁵Dy, ¹⁵⁵Eu, ¹⁵³Gd, ¹⁹⁸Au, ³H, ¹⁶⁶Ho, ^{113m}In, ^{115m}In, ¹²³I, ¹²⁵I, ¹³¹I, ¹⁸⁹Ir, ¹⁹¹Ir, ¹⁹²Ir, ¹⁹⁴Ir, ⁵²Fe, ⁵⁵Fe, ⁵⁹Fe, ¹⁷⁷Lu, ¹⁰⁹Pd, ³²P, ²²⁶Ra, ¹⁸⁶Re, ¹⁸⁸Re, ¹⁵³Sm, ⁴⁶Sc, ⁴⁷Sc, ⁷²Se, ⁷⁵Se, ¹⁰⁵Ag, ⁸⁹Sr, ³⁵S, ¹⁷⁷Ta, ¹¹⁷mSn, ¹²¹Sn, ¹⁶⁶Yb, ¹⁶⁹Yb, ⁹⁰Yt, ²¹²Bi, ¹¹⁹Sb, ¹⁹⁷Hg, ⁹⁷Ru, ¹⁰⁰Pd, ^{101m}Rh, and ²¹²Pb.

9. A kit for a direct immunohistochemical or immunocytochemical assay for cancer detection, comprising:
(a) an antibody that specifically binds to tenascin, said antibody labeled with a detectable group; and
(b) instructions for use thereof in the direct immunohistochemical or immunocytochemical assay.

10. The kit of claim 9, wherein the kit further comprises control samples, wherein the control samples are positive, negative or both.

11. A kit for an indirect immunohistochemical or immunocytochemical assay for cancer detection, comprising:
(a) a primary antibody that specifically binds to tenascin;
(b) a secondary antibody that specifically binds to said primary antibody, said secondary antibody labeled with a detectable group; and
(c) instructions for use thereof in the indirect immunohistochemical or immunocytochemical assay.

12. The kit of claim 11, wherein the kit further comprises control samples, wherein the control samples are positive, negative or both.
